(19) United States
(12) Patent Application Publication
(10) Pub. No.: US 2017/0000636 A1
(43) Pub. Date: Jan. 5, 2017

(54) IMPLANTABLE PROSTHETIC DEVICE FOR WEIGHT LOSS IN AN OBESE OR OVERWEIGHT PATIENT COMPRISING AN INFLATABLE GASTRIC BALLOON AND A DUODENAL PROSTHESIS

(71) Applicant: ASSISTANCE PUBLIQUE - HÔPITAUX DE PARIS, Paris Cedex 10 (FR)

(72) Inventors: GIANFRANCO DONATELLI, PARIS (FR); FREDERIC PRAT, SCEAUX (FR)

(21) Appl. No.: 15/037,572

(22) PCT Filed: Nov. 18, 2014
(86) PCT No.: PCT/EP2014/074933

§ 371 Date: Sep. 21, 2016

(30) Foreign Application Priority Data
Nov. 18, 2013 (FR) 1361248

(51) Int. Cl. A61F 5/00

(52) U.S. Cl. CPC A61F 5/003; A61F 5/0073; A61F 5/0076

(57) ABSTRACT

The invention relates to a prosthetic device intended for being implanted in a patient's gastrointestinal tract, said device comprising:
an inflatable gastric balloon, the volume of which is modulatable;
a duodenal prosthesis comprising a pre-pyloric flange and a flexible tubular body.

According to the invention, such a device is characterized in that it comprises a system for coupling said balloon to said duodenal prosthesis; said duodenal prosthesis is compressible and expandable in a radial direction; said prosthesis has a wall that is fixedly attached, on at least one part of its length, to a helical frame and said helix has an irregular pitch.

### 1. FIELD OF THE INVENTION

The field of the invention is that of prosthetic devices that are to be implanted in the gastrointestinal tract.

More specifically, the invention relates to a prosthetic device to be implanted in a patient's gastrointestinal tract, the prosthetic device comprising an inflatable and modulatable balloon for the stomach and an endo-prosthetic device to be placed in the patient's duodenum.

### 2. PRIOR ART

The World Health Organization (WHO) estimates that about one billion individuals in the world are currently overweight. Three hundred million of them are clinically obese with a Body Mass Index (BMI) of over 30 kg/m²: http://www.who.int/dietphysicalactivity/media/en/gsfs obesity.pdf). It would seem that the current trend is for these figures to increase.

The notions of obesity and overweight are commonly assessed through the body mass index (BMI) which is the ratio between the weight in kilograms and the square of the height in meters: BMI=mass (kg)/height² (m). A patient is considered to be overweight when his or her BMI is greater than or equal to 25. Obesity is declared when the BMI is greater than or equal to 30 and morbid obesity is determined for a BMI greater than or equal to 35 or 40.

Obesity and overweight have various etiologies and are also a source of different problems: joint-related disorders, breathlessness, difficulty of movement, excessive fatigue, depression due to poor self-image, feelings of being rejected by society, etc. In addition to these problems, obesity and overweight are non-negligible factors of risk for the patient's health and especially for cardio-vascular diseases, hypertension, certain forms of cancer and type 2 diabetes. In addition, the care required for these patients and their pathologies amounts to about 2% to 7% of expenditure by health-care systems in the developed countries. Obesity and overweight have therefore become global public health problems.

Type 2 diabetes, also commonly called acquired diabetes or diabetes mellitus, is not insulin-dependent, i.e. it is not caused by an absence of production of insulin by the Langerhans islets in the pancreas or by a production of inactive insulin. Type 2 diabetes results from the fact that, under the effect of excessive stimulation, the pancreas produces increasing amounts of insulin until the cells are exhausted. When the quantity of insulin produced is not sufficient to regulate the patient's glycemia, this patient goes into a state of hyperglycemia. It can be many years between the onset of the disease and the appearance of the first symptoms. A regular increase in the blood sugar level leads to glucotoxicity. This glucotoxicity is itself an aggravating factor in type 2 diabetes and cardiovascular diseases. Type 2 diabetes can also be characterized by micro-angiopathic and macro-angiopathic lesions in the pancreas.

At present, the standard treatment for obesity and overweight is chiefly surgery. The purpose is to help the patient to rapidly lose weight and accelerate this process. It is of course necessary give the patient with long-term treatment by dealing with the causes of his overweight and obesity, for example by teaching him to live healthily and/or by providing psychological support. It remains however necessary to make the patient rapidly lose weight in order to limit the risks of his developing cardiovascular pathology, or having a heart attack or suffering the range of pathologies that have begun to develop. It is also necessary to quickly normalize his diabetes. This surgical treatment is of two types:
the constriction of the stomach, an operation known as “vertical gastroplasty” or “sleeve gastrectomy”; or
the pyloric bypass in which a part of the stomach is directly connected to the small intestine in order to limit the absorption of food

The constriction of the stomach can be done by various surgical procedures. There is for example the known method of placing a gastric band which is an implantable device used to regulate the influx of food into the stomach. A gastric band takes the form of a belt-like device which the surgeon inflates by injecting a little liquid regularly. The gastric bands can be a cause a painful sensation or discomfort. The presence of a gastric band implies great constraints for the patient: it is no longer possible to drink while eating, food intake has to be reduced and divided into fractions spread over the whole day, it is no longer possible to engage in contact sports, food has to be cut into very small pieces, the patient has to masticate lengthily, etc. In addition, obstructions of the band can occur when the food is a little too big to pass through the orifice. Finally, in 10% of cases, it has been observed that the gastric band, originally placed outside the stomach, at the gastro-esophagal junction, migrates through the wall of the stomach to reach the stomach lumen. In other words, the band gets imprisoned in the gastric tissue and passes through the tissue so as to be partially placed in the lumen. This imprisoning of the band is accompanied by injuries, tissue perforations, inflammation and even partial necrosis in the injured area. The withdrawal of the band requires a major and extremely delicate surgical operation on the patient because it involves releasing the band from the tissue, removing the injured parts and stitching back the tissues.

Hence, although the gastric band provides for appropriate weight loss in patients, this device can cause considerable problems and can also be a bad experience for the patient.

There is also the known operation of sleeve gastrectomy in which the volume of the stomach is reduced by stapling the vertical wall of the stomach longitudinally so as to leave only a narrow tube (calibrated on a probe during the operation) at this position. The excess part of the stomach is then removed.

In the stomach bypass, the volume of the stomach is reduced by isolating a small pocket situated in the upper part of the stomach by stapling. The greater part of the volume of the stomach no longer communicates with the esophagus. This small isolated pocket is then directly connected to a loop in the small intestine, thus limiting the absorption of food by bypassing a part of the small intestine. This procedure ensures faster and more complete slimming than with a gastric band (K. S.Gersin et al., Gastrointestinal Endoscopy 2010, 71(6): 976-982). However, because of poor absorption, patients are often undernourished and vitamin supplements have to be taken for life.

It has been observed that, after these operations, patients suffering from type 2 diabetes slim down or benefit from them, and achieve spontaneous recovery from their diabetic disorder since diabetes can be reversible through weight loss. The risks of developing cardiovascular pathologies are also reduced (A. Genco et al., Obes. Surg., 2013, 23: 515-521).

However, since treatment for diabetes is a lifelong affair, compliance with the treatment declines over time. The patient therefore requires regular follow-up. In addition, not all patients can undergo surgical operations. Some of them are fearful of anesthesia and hence refuse to undergo major operations. Finally, these operations are not risk-free in themselves and can be accompanied by a sensation of discomfort or pain.

Prosthetic devices, implantable by endoscopy, have therefore been conceived in order to overcome the risks related to general anesthesia and surgery.

A first type of device is the intra-gastric balloon which is a pouch whose volume can be adjusted by the surgeon. The balloon partially fills the stomach, the apparent volume of the stomach is reduced and the patient gets a sensation of being sated more quickly. This treatment is efficacious. However, certain complications can arise: it happens indeed that the acidity of the stomach damages the material forming the balloon and the balloon gets pierced and gradually deflated. Certain studies have especially reported a rate of migration of the balloon by about 7% (A. Genco et al., Obes. Surg., 2013, 23: 953-958).

A recent development has made it possible to propose treatment for obese and overweight patients who cannot or do not wish to undergo heavy surgery. This is the EndoBarrier®. The EndoBarrier® is an endo-prosthetic device which takes the form of a flexible silicone sleeve with a pre-pyloric flange. The flexible sleeve is used to line the interior of the pylorus and the duodenum. The pre-pyloric flange is a classic prosthetic flange made of metal, comprising small metal barbs or hooks on its outer periphery. These barbs are used to anchor the ring in the internal wall of the stomach in order to maintain the EndoBarrier® in position and prevent its migration. Food therefore travels from the stomach, through the prosthesis, and the silicone sleeve makes it possible to avoid the absorption of food by the pylorus by preventing contact between the food and the intestinal wall.

Although efficacious, this technique is not free of drawbacks. The first problem is related to the migration of the prosthesis. Sometimes, the barbs get unhooked from the wall of the stomach. The prosthesis then tends to migrate into the patient's gastrointestinal tract under the effect of the alimentary bolus and intestinal peristalsis. Another drawback encountered with the EndoBarrier® is related to the presence of barbs and their hooking into the internal wall of the stomach. It can happen that the wall of the pylorus get perforated by the barbs situated in the pre-pyloric flange of the prosthesis, thus causing pain, bleeding, infection (formation of abscesses) and a risk of peritonitis, which can have a fatal outcome. Another risk linked to the use of the EndoBarrier® is related to the flexibility of the silicone sleeve. It often happens that the sleeve gets twisted, blocking the passage of the food. There is therefore a phenomenon similar to intestinal obstruction, requiring quick surgery on the patient. Again, certain patients find it very difficult to bear the presence of the prosthesis and complain of pain, making it necessary to remove the prosthesis (R. Schouten et al., Annals of Surgery, 2010, 251 (2): 236-243).

Finally, surgical operations require general anesthesia with intubation of the patient for one to three hours, or even five hours in the event of complications.

### 3. GOALS OF THE INVENTION

The invention is aimed especially at overcoming these drawbacks of the prior art.

In particular, it is a goal of the invention, in at least one embodiment, to provide a device for obese or overweight patients who cannot or do not wish to undergo an operation.

More specifically, it is a goal of the invention, in at least one embodiment, to provide a prosthetic device designed to be placed in a patient's gastrointestinal tract, making it possible to promote weight loss in a patient.

It is another goal of the invention to implement a prosthetic device which makes it possible, in at least one embodiment, to treat type 2 diabetes or at the very least to limit the development of type 2 diabetes.

It is yet another goal of the invention, in at least one embodiment, to propose such a device that is easy to implant.

It is also a goal of the invention, in at least one embodiment, to propose a prosthetic device that does not require general anesthesia with intubation or major surgery in order to be implanted in the patient's body.

### 4. SUMMARY OF THE INVENTION

These goals as well as others that shall appear here below are attained by means of a prosthetic device to be implanted in a patient's gastrointestinal tract, said device comprising:
an inflatable gastric balloon, the volume of which is modulatable;
a duodenal prosthesis comprising a pre-pyloric flange and a flexible tubular body.

According to the invention, such a device is characterized in that it comprises a system for coupling said balloon to said duodenal prosthesis, in that said duodenal prosthesis is compressible and expandable in a radial direction and in that said prosthesis has a wall that is fixedly attached, on at least one part of its length, to a helical frame.

The originality of the invention is two-fold:
the system for coupling the duodenal prosthesis to the balloon limits or even prevents the risks of migration, perforation and infection; and
the helical frame of the duodenal prosthesis prevents intestinal obstruction by keeping the tubular element constantly open. At the same time, the prosthesis is radially deformable in order to facilitate placement and removal by endoscopy.

The device according to the invention therefore makes it possible to combine the effects of restricting the gastric volume and of malabsorption in order to accelerate weight loss by the patient. This rapid weight loss makes it possible, at a first stage, to slow down or stop the development of an initial pathology or at the very least to reduce the probability of the development of such a pathology. For example, this rapid weight loss stabilizes type 2 diabetes or, when the patient has the benefit of being treated in time, reverses the development of type 2 diabetes. This also applies to other types of dysfunction such as arterial hypertension, sleep apnea, etc.

The coupling system in which the duodenal prosthesis is linked to the balloon averts the presence of hooks in the pre-pyloric flange. The absence of hooks in the pre-pyloric flange prevents the prosthesis from getting fixed to the inner wall of the stomach. The risks of perforation and infection are therefore avoided. It will also be understood that since the duodenal prosthesis is attached to the gastric balloon, and since the balloon, when inflated by the surgeon, has a diameter greater than that of the pylorus, the risks of migration are averted or at any rate considerably limited.

The helical frame of the prosthesis keeps the tubular body open. Prior-art prosthetic devices have a flexible tubular body without structure, for example a sock. Under the influence of intestinal peristalsis and the passage of food, this tubular body tends to form folds and get twisted so that the passage of food and gases is totally blocked. This phenomenon is similar to that of intestinal obstruction. The result of this is pain and vomiting. The helical frame fixedly attached to the wall of the tubular body keeps it open. It enables the tubular body to withstand the mechanical stresses that are exerted on it. The risk of obstruction is thus prevented or at least greatly limited.

Another advantage brought by the invention is that the patient does not need to be intubated during general anesthesia and that the time spent under anesthesia can be reduced. In addition, the rapid slimming of the patient provided by the device according to the invention is accessible to the most obese patients, for example those having a BMI greater than or equal to 60.

Preferably, the helical frame is fixedly attached to the wall of the tubular body. The external wall of the tubular body is defined as being the wall in contact with the intestinal tissues. The internal wall is the wall in contact with the alimentary bolus. The helical frame can be fixedly attached to the internal wall of the tubular body or to the external wall of the tubular body or it can be included in the wall. Preferably, the helical frame is fixedly attached to the internal wall or included in the wall. Thus, the helical frame is prevented from being put into contact with the intestinal wall, and this prevents friction. In an even more preferred way, the helical frame is included in the wall of the tubular body. The wall of the tubular body can be thin and its thickness can be lower than or equal to 1 mm.

The helical frame of the duodenal prosthesis also makes it possible for the device according to the invention to be deformable in a radial direction, i.e. the device according to the invention can be stressed in a catheter so that it can be placed and removed by endoscopy. The radial direction is taken with reference to the axis of the prosthesis, which is parallel to the axis of the transit of food through the prosthesis.

Endoscopy averts the need for general anesthetic which is heavy and prolonged, and entails risks for patients with enfeebled cardiovascular and respiratory systems as is the case with certain obese or overweight patients. Endoscopy also averts the need for deep incisions which take long to heal, are painful and can get infected or be accompanied by hernia. Finally, endoscopy is a speedy technique, well-mastered and more reassuring for the patient.

This device can be proposed to any type of patient, whether obese or overweight. More specifically, this device can be proposed to a patient whose BMI is greater than or equal to 30, if he is suffering from type 2 diabetes. Preferably, the period of time during which the device according to the invention remains implanted within a patient's gastrointestinal tract ranges from four months to twelve months, preferably six months to twelve months. The device according to the invention is therefore intended for removal and should not be implanted for the patient's entire life. It is therefore a transitional or temporary device.

More precisely, the device according to the invention, can be proposed to patients having a BMI greater than or equal to 30 when they shows signs of co-morbidity (arterial hypertension, sleep apnea, breathlessness, pre-diabetic condition etc.) and can be systematically proposed to patients whose BMI is greater than or equal to 35. It must be noted that, at present, the prior-art devices are systematically proposed only to patients with a BMI of 40, or with a BMI greater than 35 with co-morbidity. The device according to the invention therefore broadens the category of patients who can benefit from it.

Advantageously, the helical pitch of said helical frame is irregular. The term “helical pitch” is understood as the value of the axial advance of the generatrix of the helix in one turn. The irregularity of the helical pitch gives a degree of flexibility to the duodenal prosthesis so that it can get sufficiently curved to adapt to the anatomy of the patient's gastrointestinal tract.

Preferably, said pitch of the helix increases from the pre-pyloric flange to the distal extremity of the tubular body. The term “proximal” extremity or “proximal” part is understood to mean the extremity or the part of the device that is on the mouth side. The “distal” extremity or “distal” part of the device designates the extremity or part of the device that is closer to the anus. In other words, the helix forming the helical frame starts from the pre-pyloric flange. The helical pitch can be small in order that the flange may be more rigid than the rest of the duodenal prosthesis. In a first variant of the invention, the helical pitch can then gradually increase so as to give increasing flexibility to the tubular body. In a second variant, the helical pitch at the position of the tubular body can be different from the pitch at the pre-pyloric flange but is regular all along the tubular body.

Advantageously, the duodenal prosthesis has no hooks which would be used to affix it to the patient's digestive tissue. This characteristic prevents or at the very least limits the risk of perforation and hemorrhage of the gastric wall and it therefore also limits the risks of bleeding and infection associated therewith. A perforation is a surgical emergency. This characteristic also facilitates the removal of the device according to the invention.

In one advantageous embodiment, the coupling system is irreversible. Thus, it is easy to withdraw the device according to the invention by seizing it through the upper endoscopic channel and withdrawing the unit in one action. The gastric balloon is deflated and the unit is withdrawn in one single operation. This characteristic further limits the risks of migration of the prosthesis in the gastrointestinal tract.

In one particularly advantageous embodiment, the coupling system makes it possible, when pulled on during the withdrawal of the gastric balloon, to prompt the withdrawal of the prosthesis.

Preferably, the coupling system comprises means of irreversible attachment. Such means can be chosen from the following: clip-on system, spring clips, a system for fitting together, a harpoon-catheter pair, a ring associated with an endoscopic clip, etc.

In one promising embodiment, said helical frame extends on a portion of the total length of the duodenal prosthesis ranging from 50 to 85%, preferably 75%. Preferably, the helical frame extends from the pyloric bulb up to the first jejunal loop.

Preferably, the helical armature extends from the proximal extremity of the pre-pyloric flange. Thus, the distal portion of the tubular body of the duodenal prosthesis is left free without a helical frame. Although this distal portion of the tubular body does not comprise any helical frame, the length left free is not sufficient for it to get twisted and block the intestine. Preferably, the length of the tubular body that does not include any helical frame does not exceed 15 cm and in a more preferred way, does not exceed 10 cm. It has indeed been observed by the inventors that these lengths do not enable the flexible tubular body to get irreversibly twisted. In other words, this free part of the tubular body without a helical frame is too short to prompt any obstruction.

In one promising embodiment, the inflatable gastric balloon and/or the duodenal prosthesis are constituted entirely or partly by a biocompatible material, such as silicone, polyurethane, nylon, polyvinyl chloride, urethane, polyamide, polyester, or a combination of at least two of these materials.

These materials have the advantage of being well tolerated by patients, withstanding the environment of the gastrointestinal tract and being resistant to sterilization and to mechanical stresses. Preferably, the gastric balloon and/or the duodenal prosthesis are made of silicone. Even more preferably, the gastric balloon and/or the duodenal prosthesis are entirely constituted by the same material.

Advantageously, said helical frame is made of a biocompatible metallic material, preferably an alloy of nickel and titanium. These materials have the advantage of being well tolerated by patients. In addition, they prove to be particularly resistant to the surrounding acid/base environment and to constant contact with partially digested food. What is more, these materials or alloys can be used to produce shape memory materials. They are therefore particularly promising for the designing of expansive and compressible prostheses. More particularly, through materials of this type combined with the helical frame, the prosthetic devices can be compressed in a catheter which will be introduced through the patient's natural cavities. These prosthetic devices are delivered to the very location of the junction between the pylorus and the proximal portion of the duodenum on a guide wire under endoscopic and radioscopic control. The release of the prosthesis in the lumen of the gastrointestinal tract results in an immediate deployment of this prosthesis. This deployment is permitted by the use of a shape memory material. This means that the prosthesis finds its initial deployed shape when the stresses exerted by the catheter disappear.

In one advantageous embodiment, the gastric balloon furthermore comprises an inflating catheter. The inflating catheter is planned in the upper part of the balloon, said upper part of the balloon being the one oriented towards the esophagus. The inflating catheter, having a length of about 30-50 cm, makes it possible to obtain variations in the volume of the balloon. The balloon can thus be gradually inflated so that the patient gets gradually used to the device and to the changes in food habits caused by the device. It is also possible to slightly inflate it if the presence of the balloon causes discomfort to the patient. Finally, inflating or deflating the balloon makes it easy to place and withdraw the balloon.

### 5. LIST OF FIGURES

Other features and advantages of the invention shall appear more clearly from the following description of a preferred embodiment given by way of a simple illustratory example and from the appended drawings, of which:

FIG. 1 presents a diagram of an embodiment of the device according to the invention implanted in a patient's gastrointestinal tract; and

FIGS. 2A and 2B present transversal and schematic views of different embodiments of an irreversible hooking system according to the invention.

### 6. DESCRIPTION OF ONE EMBODIMENT OF THE INVENTION

The device according to the invention proposes to associate the efficiency of an inflatable gastric balloon with that of a duodenal endo-prosthesis. The device according to the invention however comprises a system for coupling the duodenal prosthesis to the balloon in order to limit the risks of migration of the prosthesis and of the gastric balloon. This characteristic also makes it possible to do away with the need for hooks, thus averting the risks of perforation of the gastric wall during the lifetime of the prosthesis within the gastrointestinal tract and at the time of withdrawal of the device. The device according to the invention also partly mitigates the drawbacks of current endo-prosthetic devices by fixedly attaching the wall of the duodenal prosthesis to a helical frame on all or part of the length of said prosthesis. The helical frame makes it possible not only to endow the prosthesis with flexibility and anatomical adaptation but also keeps the tubular body open in order to prevent the phenomenon of obstruction caused by the twisting of the tubular body.

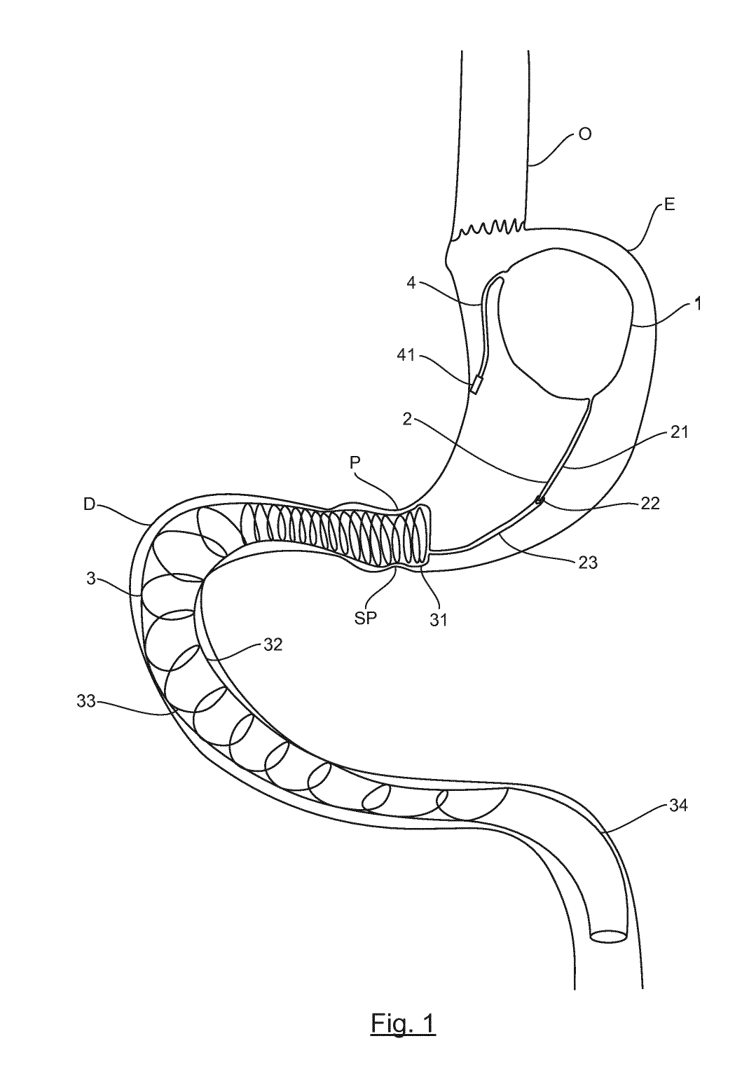

FIG. 1 presents an embodiment of the device according to the invention when it is implanted and deployed in a patient's gastrointestinal tract, the patient being an obese individual (withy BMI greater than 30) or an overweight individual (with BMI greater than 25).

The device according to the invention comprises a gastric balloon 1 connected to a duodenal prosthesis 3 by a coupling system 2.

The gastric balloon 1 comprises an inflating catheter 4 and an attachment element 22. The inflating catheter 4 enables the surgeon to inject either air or saline solution into the balloon in order to increase its volume. Preferably, the gastric balloon 1 is filled with air. It can also be filled with a colored liquid by which the patient can identify any premature damage to the balloon. The volume of gastric balloon can be modulated and can vary when it is inflated, between 400 cm³ and 750 cm³. The gastric balloon 1 has an external wall, in contact with the stomach, and an internal wall in contact with air or a saline solution. In this embodiment, the external wall of the balloon 1 is made of silicone while the internal wall is made of gold-metallized polyurethane. The inflation catheter 4 comprises at its extremity 4 a closing-off means 41 such as a one-way valve to prevent air or liquid contained in the balloon from leaking into the patient's stomach. This also enables the removal of a little air or liquid if the patient feels discomfort. It also enables a little air or liquid to be added in order to bring about gradual variations in the volume of the balloon so as to allow the patient time to get used to changes in food habits involved in the placing of such a device.

The coupling system 2 comprises an element 21 fixedly joined to the balloon 1, an element 23 fixedly attached to the duodenal prosthesis 3 and fixed attachment means 22. The elements 21 and 23 can take the form of a fine catheter, the diameter of which ranges from 2 to 5 mm, preferably about 3 mm.

Preferably, the fixed attachment means are irreversible. These means can be each constituted by each of the extremities of the elements 21 and 22.

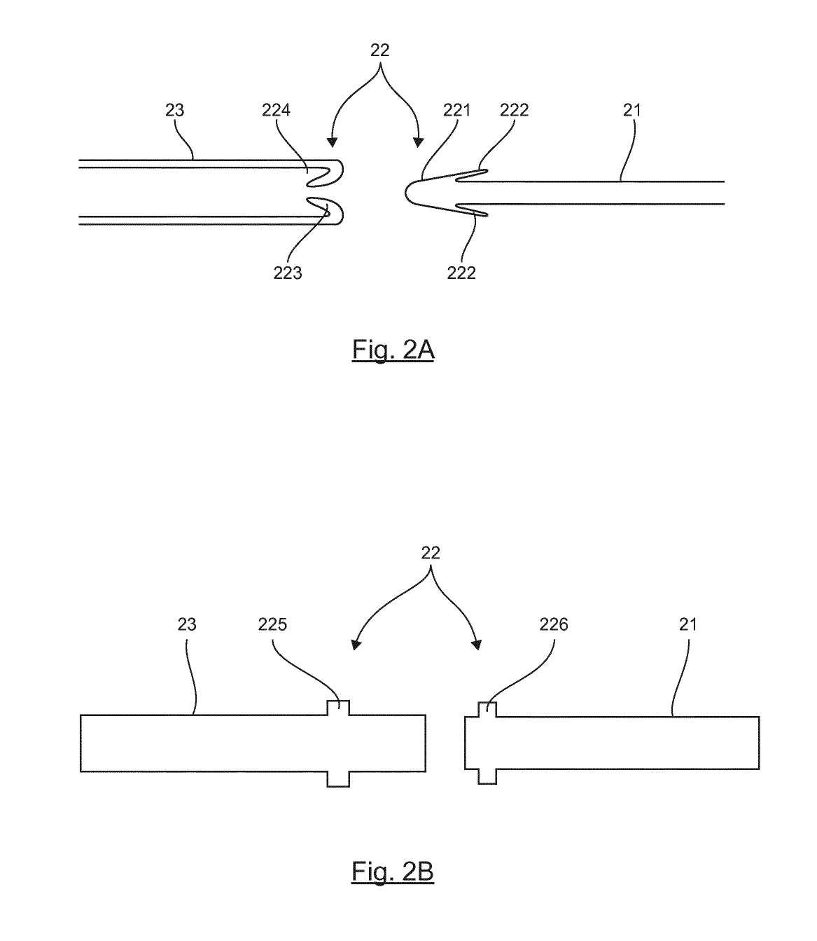

Referring to FIGS. 2A and 2B, we present different embodiments of the irreversible coupling system according to the invention.

FIG. 2A shows a cross section of a first embodiment corresponding to a harpoon-catheter system. In this first variant, the extremity of the element 21 can take the form of a harpoon that gets force-fitted into the element 23. As shown, the catheter 21 comprises at its extremity an end-piece 221 in the form of a rounded tip and two barb-like features 222. The interior of the catheter 23 comprises a hook-forming structure 223 at its extremity, on its internal periphery. Thus, when the tip 221 enters by elastic deformation into the catheter 23 through the structure 223, the barb-like features 222 get lodged in the recess 224 and are retained by the structure 223.

FIG. 2B shows a second embodiment of the coupling system 2 with a fitting-in means. In this embodiment, the element 21 can comprises one or more collars 226 on its external periphery intended to cooperate with one or more internal peripheral grooves 225 at the element 23. Those skilled in the art will understand that the characteristics of the elements 21 and 23 can be reversed.

The coupling system 2 prevents the migration of the duodenal prosthesis 3 into the gastrointestinal tract, it being understood that the once the gastric balloon is inflated, it cannot pass through the pyloric sphincter SP. It also facilitates the withdrawal of the device according to the invention from the patient's gastrointestinal tract.

The duodenal prosthesis 3 has an essentially tube-like shape: in its proximal part, it comprises a pre-pyloric flange 31, a tubular body 32 and a distal portion 34. The duodenal prosthesis 3 consists of a biocompatible material, preferably non-biodegradable such as silicone, nylon or polyurethane, which give it high flexibility and prevent the food from being absorbed in the duodenum.

The prosthesis 3 also comprises a helical frame 33 fixedly attached to the wall of the prosthesis. The helical structure gives the prosthesis 3 its deformability in a radial direction so that the prosthesis 3 can be stressed in a catheter when it is placed by endoscopy and so that the withdrawal of the endoscopy catheter enables its immediate deployment, leaving the prosthesis 3 in intimate contact with the internal wall of the gastrointestinal tract.

The helical frame 33 also gives flexibility to the prosthesis so that, on the one hand, it adapts to the anatomy of the gastrointestinal tract and, on the other hand, resists intestinal peristalsis. It also keeps enables the prosthesis to be kept open. Indeed, the prior-art prostheses, which have no helical frame, tend to fold and get twisted until they totally obstruct the passage of food. The frame 33 which is fixedly joined to the wall of the prosthesis avoids such drawbacks.

The pre-pyloric flange 31 is intended for deployment in the stomach, at the pylorus upstream to the pyloric sphincter SP. Its diameter ranges from 25-35 mm, so that the diameter of the flange is greater than that of the pyloric sphincter and prevents the migration of the prosthesis into the gastrointestinal tract. Preferably, the pre-pyloric flange 31 has no metal hook. Through the coupling system 2, it is not necessary to provide for hooks at the prosthesis itself to get it directly hooked to the gastric tissue. Thus, the problems of irritation, perforation and infection are avoided or at the very least greatly limited. The main body 32 of the prosthesis 3 has a constant diameter smaller than that of the flange 31, ranging from 22 to 24 mm.

The total length of the prosthesis 3 can range from 40 cm to 70 cm. Preferably, the helical frame extends from the proximal extremity of the flange on about 75% to 85% of the total length of the prosthesis, giving a length of 30 cm to60 cm. This means that the distal part 34 of the prosthesis is not fixedly attached to a helical frame on about 10 cm. In other words, the distal portion 34 without frame is left free. The inventors have indeed observed that this length is not sufficient for the material to fold or get twisted and thus induce intestinal obstruction.

In this embodiment, the helical pitch is irregular: it is small at the level of the flange and the proximal part of the prosthesis. It increases evenly along the tubular body.

As represented in FIG. 1, the gastric balloon 1 is released in a patient's stomach E at the level of the fundus beneath the esophagus 0. The duodenal prosthesis 3 extends from the pylorus P and along the duodenum D. The surgeon attaches the balloon 1 in vivo to the prosthesis 3 by means of the system for coupling 2.

### 7. VARIANTS

Other variants of the coupling system can be envisaged. For example, it is possible to provide for a ring on the pre-pyloric flange or the catheter 23 can be made to end in a loop. In this case, the catheter 21 will be terminated by a clamp so as to get fixed into the ring. In another variant, the catheter 23 is terminated by a clamp. When the clamp is made to penetrate the catheter 21 by plastic deformation, the arms of the clamp separate once they are in the catheter 23 and exert a force against the internal wall of the catheter 23 preventing them from going out.

1. Prosthetic device to be implanted in a patient's gastrointestinal tract, said device comprising:
an inflatable gastric balloon (1) the volume of which is modulatable;
a duodenal prosthesis (3) comprising a pre-pyloric flange (31) and a flexible tubular body (32);
characterized in that:
said device comprises a system (2) for coupling said balloon (1) to said duodenal prosthesis (3) ;
said duodenal prosthesis (3) is compressible and expandable in a radial direction;
said prosthesis (3) has a wall that is fixedly attached, on at least one part of its length, to a helical frame (33) and
said helix has an irregular pitch.

2. Device according to claim 1, wherein said pitch of said helix increases from the pre-pyloric flange (31) to the distal extremity of the tubular body (32).

3. Device according to one of the preceding claims, wherein the duodenal prosthesis (3) is without hooks.

4. Device according to one of the preceding claims, wherein the system (2) for coupling comprises means (22) of irreversible fixed attachment means.

5. Device according to claim 4, wherein said means of irreversible attachment are chosen from among a clip-on system, spring clips, a system for fitting together (225, 226), a harpoon-catheter system (221,222, 223, 224).

6. Device according to one of the preceding claims, wherein said helical frame (33) extends on a portion of the total length of the duodenal prosthesis ranging from 50% to 85%, preferably 75%.

7. Device according to one of the preceding claims, wherein said modulatable gastric balloon (1) and/or the duodenal prosthesis (3) are constituted entirely or partly by a biocompatible material, such as silicone, polyurethane, nylon, polyvinyl chloride, urethane, polyamide, polyester, or the combination of at least two of these materials.

8. Device according to one of the preceding claims, wherein said helical frame (33) is made of a biocompatible metallic material, preferably an alloy of nickel and titanium.

9. Device according to one of the preceding claims, the gastric balloon (1) comprises an inflating catheter (4).
